(19) United States
(12) Patent Application Publication
(10) Pub. No.: US 2025/0192599 A1
(43) Pub. Date: Jun. 12, 2025

(54) INTEGRATED, SHAREABLE POWER SUPPLY FOR MEDICAL PROCEDURE DEVICES AND METHODS OF MAKING AND USING SAME

(71) Applicants: Mark T. McBride, Coronado, CA (US); John Roughneen, Dennis, MA (US); Glenn Butler, Holliston, MA (US)

(72) Inventors: Mark T. McBride, Coronado, CA (US); John Roughneen, Dennis, MA (US); Glenn Butler, Holliston, MA (US)

(21) Appl. No.: 18/908,953

(22) Filed: Oct. 8, 2024

Related U.S. Application Data
(60) Provisional application No. 63/606,636, filed on Dec. 6, 2023.

(51) Int. Cl. H02J 7/34; A61B 90/30; A62B 18/00; A62B 18/08

(52) U.S. Cl. CPC H02J 7/34; A61B 90/30; A62B 18/003; A62B 18/08

(57) ABSTRACT

Systems, methods, and other embodiments for an integrated, shareable power supply system for medical procedure devices, including a helmetless support and ventilation system, a power module located within the helmetless support and ventilation system, and an integrated, shareable power supply system located on at least one side of the helmetless support and ventilation system, wherein the integrated, shareable power supply system is electrically connected to the power module.

### CROSS-REFERENCE TO RELATED APPLICATION

This application claims benefit of U.S. Patent Application 63/606,636, filed on Dec. 6, 2023, the disclosure of which is hereby incorporated by reference in its entirety to provide continuity of disclosure to the extent such a disclosure is not inconsistent with the disclosure herein.

### FIELD OF THE INVENTION

The present invention is generally related to an integrated, shareable power supply system for medical procedure devices that can be connected to helmetless support and ventilation systems for use with surgical hoods and gowns. The system enables the user to eliminate prior power solutions and takes advantage of the additional capacity of the internal battery in the helmetless support and ventilation system. Furthermore, the system will eliminate the additional weight associated with those prior power solutions. In this manner, the power source that is provided with the helmetless support and ventilation system can then be commonly used with any medical procedure device that can be connected to the helmetless support and ventilation system.

### BACKGROUND OF THE INVENTION

Prior to the present invention, as set forth in general terms above and more specifically below, it is known that power requirements associated with lighting and augmented reality (AR) solutions for use in medical procedures can be very complex. For example, each of these lighting and AR solutions (such Augmented Reality, Mixed Reality, and Virtual Reality solutions) has have their own direct current (DC) power supplies. Also, each one of these DC power supplies needs to be worn by the operator (such as medical personnel performing a medical procedure) in unique locations from on the waist, around the neck or back, or even on the head. Since these DC power supplies can become very cumbersome, these DC power supplies can create challenges for cable management, not to mention the additional weight of each of the DC power supplies. Therefore, it would be desirable to provide an integrated, shareable power supply system for medical procedure devices that can be connected to helmetless support and ventilation systems for use with surgical hoods and gowns, wherein the integrated, shareable power supply system would enable the user to eliminate those known power solutions and take advantage of the additional capacity of the internal battery in the helmetless support and ventilation systems. Furthermore, it would be desired to be able to reduce or eliminate the additional weight associated with those known DC power solutions. Finally, it would be desired to provide a helmetless support and ventilation system having a power supply that can then be commonly used with any medical procedure device that can be connected to the helmetless support and ventilation system.

It is a purpose of this invention to fulfill these and other needs in the power supply for medical devices art in a manner more apparent to the skilled artisan once given the following disclosure.

The preferred integrated, shareable power supply system for medical procedure devices, according to various embodiments of the present invention, offers the following advantages: ease of use; reduction in weight in the power supply; the ability to quickly connect the medical device to the helmetless support and ventilation system; the ability to quickly disconnect the medical device from the helmetless support and ventilation system; the ability to use the battery located within the helmetless support and ventilation system; the ability to quickly change out the battery in the helmetless support and ventilation system; and the ability to provide the helmetless support and ventilation system with a power supply that can be commonly used with the helmetless support and ventilation system and any medical procedure device that can be connected to the helmetless support and ventilation system. In fact, in many of the preferred embodiments, these advantages are optimized to an extent that is considerably higher than heretofore achieved in prior, known power supply systems for medical devices.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, isometric view of a known helmetless support and ventilation system that can be used with surgical gowns having hoods, according to one embodiment of the prior art;

FIG. 2 is a rear, isometric view of an integrated, shareable power supply system for medical procedure devices for use with a helmetless support and ventilation system, constructed according to one embodiment of the present invention;

FIG. 3 is a side, isometric view of the integrated, shareable power supply system for medical procedure devices for use with a helmetless support and ventilation system, constructed according to one embodiment of the present invention;

FIGS. 4 and 4A are isometric views of the integrated, shareable power supply system for medical procedure devices for use with a helmetless support and ventilation system, constructed according to an embodiment of the present invention;

FIG. 5 is a side view of the integrated, shareable power supply system for medical procedure devices for use with a helmetless support and ventilation system, constructed according to one embodiment of the present invention;

FIG. 6 is a rear view of the integrated, shareable power supply system for medical procedure devices for use with a helmetless support and ventilation system, constructed according to one embodiment of the present invention;

FIGS. 7 and 7A are rear, isometric views of the integrated, shareable power supply system for medical procedure devices for use with a helmetless support and ventilation system, with the integrated, shareable power supply system being located on different sides of the helmetless support and ventilation system, constructed according to an embodiment of the present invention; and

FIG. 8 is a schematic, isometric, rear view of the integrated, shareable power supply system for medical procedure devices for use with a helmetless support and ventilation system with a yoke module being attached, according to an embodiment of the present invention.

### DETAILED DESCRIPTION OF THE PREFERRED EMBODIMENTS OF THE INVENTION

In order to address the shortcomings of the prior, known power supplies for medical devices, it would be desirable to provide an integrated, shareable power supply system for medical procedure devices that can be connected to helmetless support and ventilation systems for use with surgical hoods and gowns, wherein the integrated, shareable power supply system would enable the user to eliminate those known power solutions and take advantage of the additional capacity of the internal battery in the helmetless support and ventilation systems. Furthermore, it would be desired to be able to reduce or eliminate the additional weight associated with those known DC power solutions. Finally, it would be desired to provide the helmetless support and ventilation system having a power supply can then be commonly used with any medical procedure device that can be connected to the helmetless support and ventilation system.

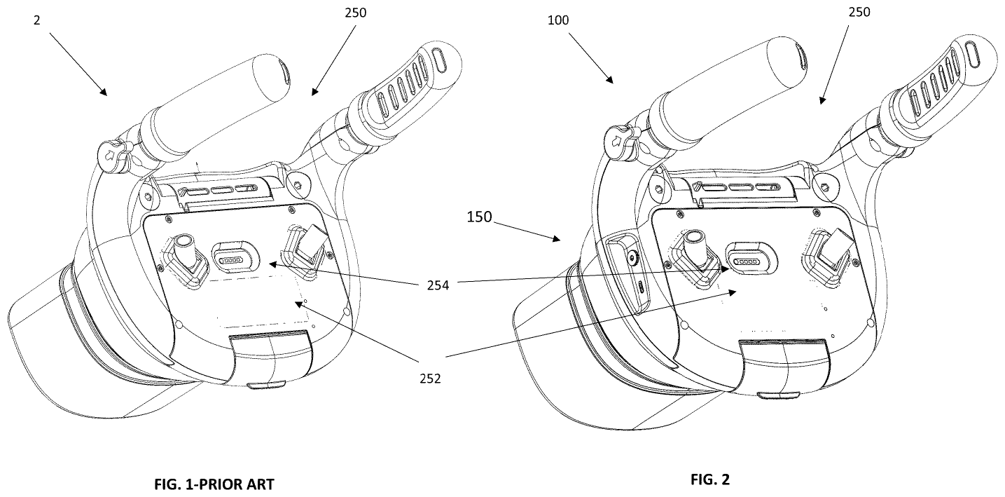

Referring now to FIG. 1, there is illustrated a known helmetless support and ventilation system 2 for use with surgical hoods and gowns (not shown). As shown in FIG. 1, helmetless support and ventilation system 2 includes, in part, power module 250. Power module 250 includes, in part, battery 252 and battery recharging port 254. Preferably, battery 252 is a conventional, rechargeable battery such as a lithium-ion battery or the like that is capable of providing sufficient power to the various devices (not shown) that are electrically connected to helmetless support and ventilation system 2 such as an air flow generation module, electric motors, and a printed circuit board (PCB) module for an extended period of time such as 6-8 hours.

### Integrated, Shareable Power Supply System

With respect to FIG. 2, there is illustrated a novel integrated, shareable power supply system 100 for medical procedure devices that can be connected to helmetless support and ventilation system 2 for use with surgical hoods and gowns (not shown). As shown in FIG. 2, helmetless support and ventilation system 2 includes, in part, power module 250. Power module 250 includes, in part, battery 252 and battery recharging port 254. It is to be understood that power module 250 is similar to power module 250, as discussed above.

As further shown in FIG. 2, integrated, shareable power supply system 100 also includes, in part, power supply assembly 150. In one embodiment, power module 250 is electrically connected to the power supply assembly 150 in order to provide the necessary electrical power to any devices such as head lamps, computing devices, AR systems (such Augmented Reality, Mixed Reality, and Virtual Reality systems) or the like (not shown) that are connected to the integrated, shareable power supply system 100 through power supply assembly 150.

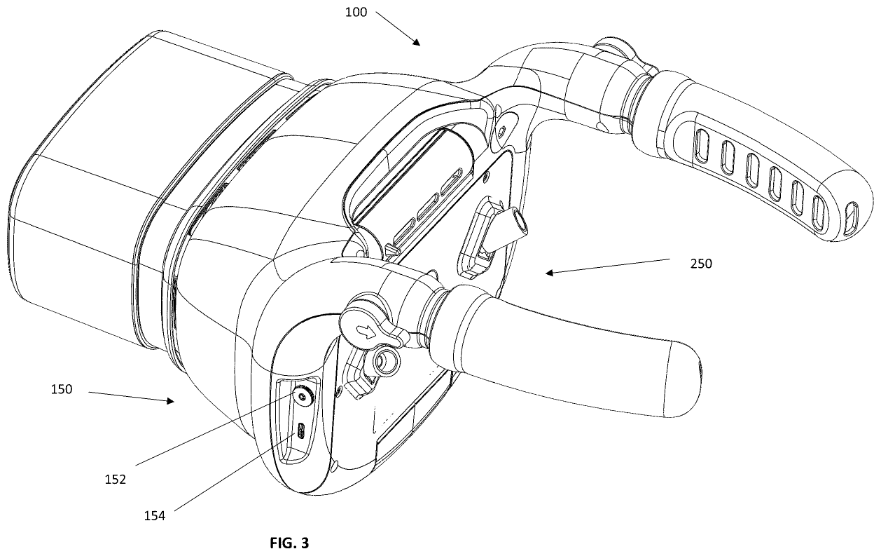

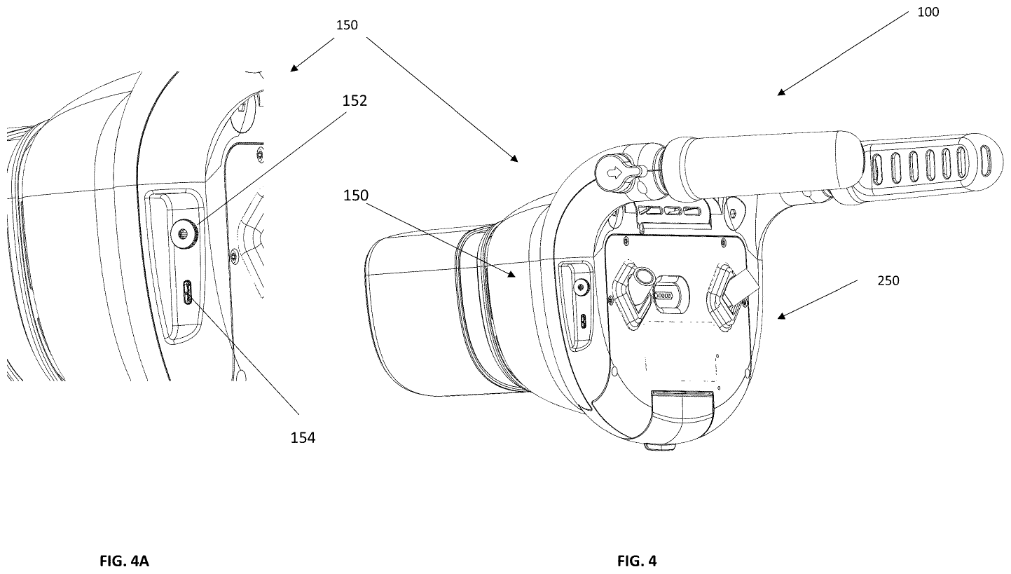

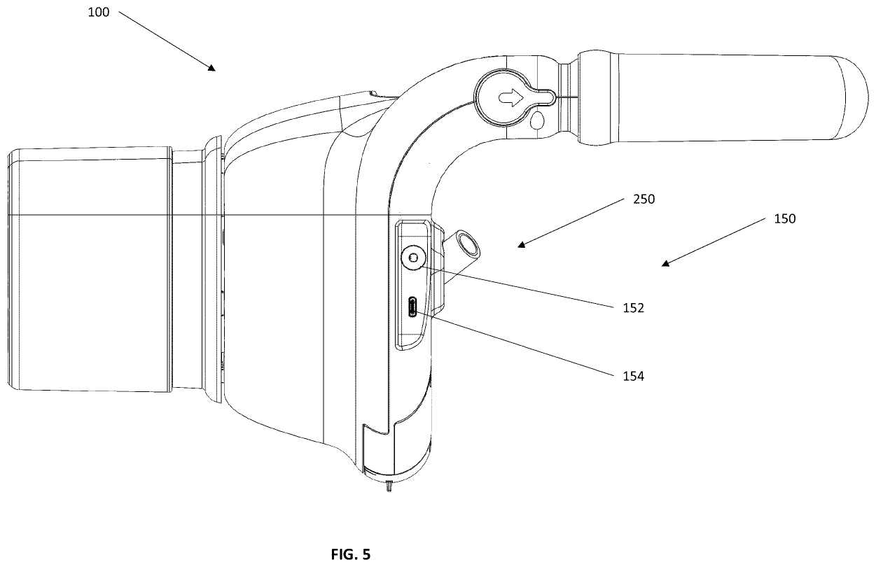

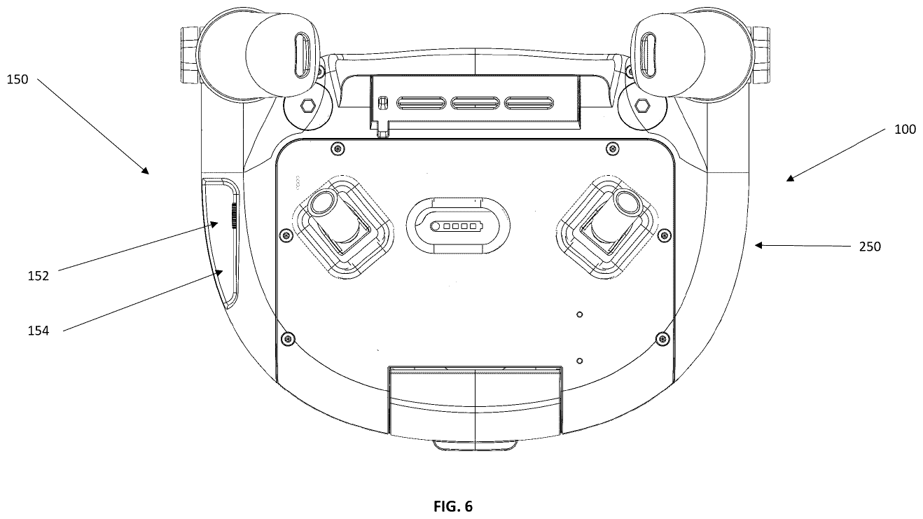

With respect to FIGS. 3-6, there is illustrated integrated, shareable power supply system 100 having power supply assembly 150. In particular, power supply assembly 150 includes, in part, headlamp (lighting) device power port 152 and USB-C or other similar electrical device power port 154. As discussed above, power module 250 is electrically connected to the power supply assembly 150 in order to provide the necessary power to any device (not shown) that is connected to the integrated, shareable power supply system 100 through power supply assembly 150 (i.e., headlamp device power port 152 and USB-C or other similar power port 154).

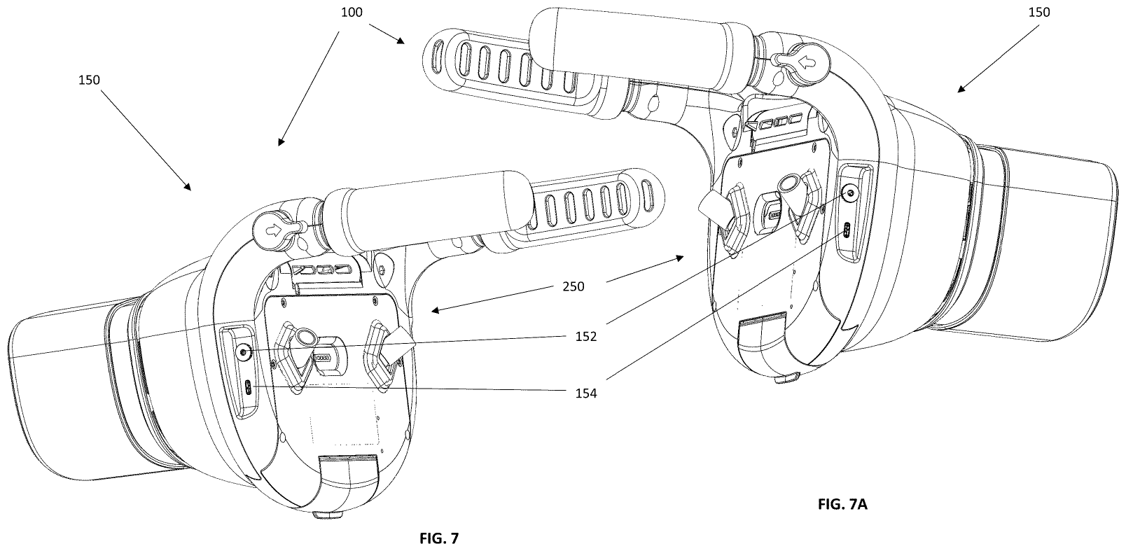

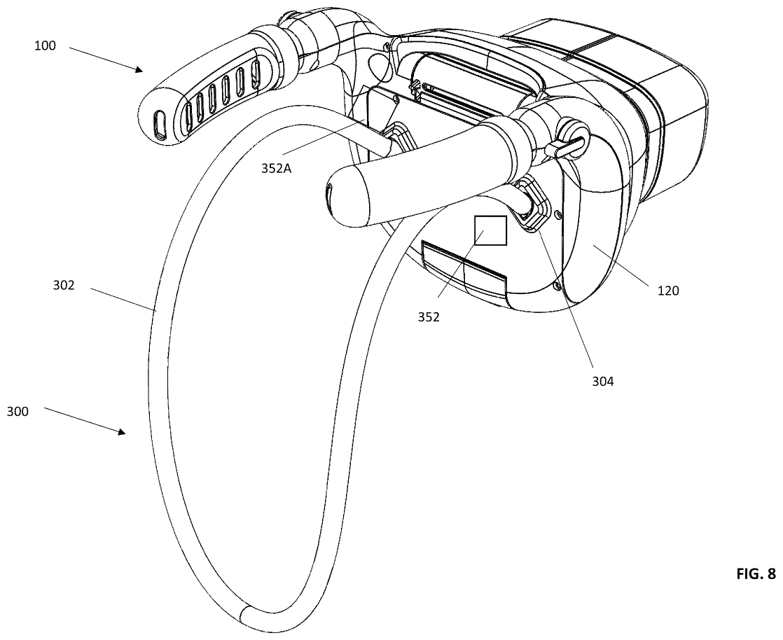

With respect to FIGS. 7 and 7A, there is illustrated the different locations on integrated, shareable power supply system 100 where power supply assembly 150 can be located. A unique aspect of the present invention is that the power supply assembly 150 can be located on the left side or the right side or both sides of the integrated, shareable power supply system 100 in order to provide the necessary power supply ports to any device (not shown) that can be connected to the integrated, shareable power supply system 100.

With respect to yoke module 300, as shown in FIG. 8, yoke module 300, includes, in part, yoke 302 and yoke connectors 304. Preferably, yoke 302 is constructed of any suitable, durable, high strength, flexible, medical grade material. Preferably, yoke connectors 304 are attached to the back of protective casing 120 of ventilation system 100.

Another unique aspect of the present invention is the use of yoke module 300. In particular, yoke module 300 can be used to assist in retaining ventilation system 100 on the shoulders of the wearer. In particular, yoke 302 is removably attached to protective casing 120 through the use of yoke connectors 304. In this manner, yoke 302 can be easily attached to and removed from protective casing 120. Furthermore, since yoke 302 is flexible, yoke 302 can be adjusted so as to fit the upper torso of the wearer so that ventilation system 100 will remain securely retained on the shoulders and the upper torso of the wearer. For example, the wearer can position the ventilation system with the yoke module 300 installed over his/her head and place the yoke module 300 on the upper torso of the wearer. The wearer can then pull/push on yoke 302 while yoke 302 is connected to yoke connectors 304 so that yoke 302 firmly contacts the upper torso of the wearer in order to assist in retaining the ventilation system 100 on the shoulders and upper torso of the wearer 6.

A unique aspect of the present invention is the use of additional (auxiliary) battery packs 352 and 352A that can be mounted on the protective casing 120 or on the yoke 302 near the yoke connectors 304 in a weight neutral position (i.e., the addition of the battery packs 352 and 352A do not create a forward or backward force on the wearer, instead the weight of the additional battery packs 352 and 352A only create a slight downward force on the wearer). These additional (auxiliary) battery packs 354 and 354A are electrically connected to the power module 250 and could be used to provide additional power to the various devices (not shown) that are electrically connected to helmetless support and ventilation system 100 such as an air flow generation module, electric motors, and a printed circuit board (PCB) module for an extended period of time in addition to the main batteries 252. In this manner, the batteries 252 are shareable from the integrated, shareable power supply system 100 and an additional battery pack 352 and/or 352A that are electrically connected to the power module 250 are also sharable from the integrated, shareable power supply system 100 and are mounted and balanced on the yoke 302.

### Using Integrated, Shareable Power Supply System

With respect to using integrated, shareable power supply system 100, attention is directed to FIGS. 2-8, wherein power supply cords (not shown) from various devices that are to be electrically connected to integrated, shareable power supply system 100 can be attached to power supply assembly 150. For example, an shareable device such as a head lamp (not shown) can be electrically connected to headlamp device power port 152. Also, a shareable device such as a computing device, AR device (such Augmented Reality, Mixed Reality, and Virtual Reality devices) or other similar device (not shown) can be electrically connected to USB-C or other similar power port 154. In this manner, the integrated, shareable power supply system 100 can be used as the primary power source to any shareable device or integrated, shareable power supply system 100 in order to augment the battery life of the shareable device (i.e. as a possible “Back-Up” power solution).

A unique aspect of the present invention is that since the various devices do not need to be electrically connected to the known power supply devices, as discussed earlier, the amount of extra weight that the user (medical personnel) needs to carry around during a medical procedure is significantly reduced or eliminated, thereby reducing the fatigue experienced by the user due to the extra weight.

Another unique aspect of the present invention is that instead of having to monitor the battery life of all of the various devices that are being used during the medical procedure, only the battery 252, 352 and/or 352A need to be monitored to ensure that the battery 252, 352 and/or 352A are operating properly. In case the battery 252, 352 and/or 352A start to run out of power, the integrated, shareable power supply system 100 can allow the user to “hot swap” or quickly change the battery 252, 352 and/or 352A without having to restart or re-boot the various devices. In this manner, the various devices can continuously operate during the medical procedure.

A further unique aspect of the present invention is that since the various devices are electrically connected to the integrated, shareable power supply system 100, a remote-control device (not shown) can be utilized to operate any or all of the devices that are electrically connected to the integrated, shareable power supply system 100. In this manner, a universal remote-control can be utilized by the user (medical personnel) to operate the various devices that are electrically connected to the integrated, shareable power supply system 100. This will eliminate the need for the user to carry around and operate multiple remote-control devices.

Another unique aspect of the present invention is the use of additional battery packs 352 and 352A that can be mounted on the protective casing 120 or on the yoke 302 near the yoke connectors 304 in a weight neutral position (i.e., the addition of the battery packs 352 and 352A do not create a forward or backward force on the wearer, instead the weight of the additional battery packs 352 and 352A only create a slight downward force on the wearer). These additional battery packs 354 and 354A could be used to provide additional power to the various devices (not shown) that are electrically connected to helmetless support and ventilation system 100 such as an air flow generation module, electric motors, and a printed circuit board (PCB) module for an extended period of time in addition to the main batteries 252. In this manner, the batteries 252 are shareable from the integrated, shareable power supply system 100 and an additional battery pack 352 and/or 352A that are electrically connected to the power module 250 are also sharable from the integrated, shareable power supply system 100 and are mounted and balanced on the yoke 302.

A still further unique aspect of the present invention is that since the helmetless support and ventilation system includes a power supply, this power supply can then be commonly used with any medical procedure device that can be connected to the helmetless support and ventilation system.

With respect to headlamp device power port 152, some of the benefits of the headlamp device power port 152 are as follows:
Powers the light off of the same battery 252, 352 and/or 352A as the main unit, thereby eliminating the need for another “pocket” battery.
Allows for the light brightness to be controlled by helmetless support and ventilation system 100:
   By a mobile application or watch.
   By the remote (for example, a single tap on up or down could control the fan speed while a double tap could control the light brightness)
   A surgeon's preference including light brightness could be loaded before surgery from the mobile application.
   In addition, severe alerts (like fan failure or low battery) could be presented to the user by flashing the light.

With respect to USB-C or other similar power port 154, some of the benefits of the USB-C or other similar power port 154 are as follows:
Can charge or run the augmented reality (AR) system using the main fan battery 252, 352 and/or 352A, which would typically be higher capacity.
The fan battery 252, 352 and/or 352A could be “hot swapped” during a surgery without having to reboot the AR system (such Augmented Reality, Mixed Reality, and Virtual Reality systems) (which could run off of its own internal battery during the swap)
Since the AR system is typically equipped with its own battery, the AR battery can be continually topped off by the battery 252, 352 and/or 352A

While the disclosed embodiments have been illustrated and described in considerable detail, it is not the intention to restrict or in any way limit the scope of the appended claims to such detail. It is, of course, not possible to describe every conceivable combination of components or methodologies for purposes of describing the various aspects of the subject matter. Therefore, the disclosure is not limited to the specific details or the illustrative examples shown and described. Thus, this disclosure is intended to embrace alterations, modifications, and variations that fall within the scope of the appended claims, which satisfy the statutory subject matter requirements of 35 U.S.C. § 101.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

Therefore, provided herein is a new and improved integrated, shareable power supply system for medical procedure devices, according to various embodiments of the present invention, offers the following advantages: ease of use; reduction in weight in the power supply; the ability to quickly connect the medical device to the helmetless support and ventilation system; the ability to quickly disconnect the medical device from the helmetless support and ventilation system; the ability to use the battery located within the helmetless support and ventilation system; the ability to quickly change out the battery in the helmetless support and ventilation system; and the ability to provide the helmetless support and ventilation system with a power supply that can be commonly used with the helmetless support and ventilation system and any medical procedure device that can be connected to the helmetless support and ventilation system.

In fact, in many of the preferred embodiments, these advantages of ease of use, lightness in weight, the ability to use the AR device (such Augmented Reality, Mixed Reality, and Virtual Reality devices) in conjunction with a face shield, the ability to use the adapter system on a variety of AR devices, portability, ease of attachment of the adapter to the AR device, ease of removal of the adapter from the AR device, the ability to provide the helmetless support and ventilation system with a power supply that can be commonly used with the helmetless support and ventilation system and any medical procedure device that can be connected to the helmetless support and ventilation system, and reduced cost are optimized to an extent that is considerably higher than heretofore achieved in prior, known power supply systems for medical devices.

What is claimed is:

1. An integrated, shareable power supply system for medical procedure devices, comprising:
a helmetless support and ventilation system;
a power module located within the helmetless support and ventilation system; and
an integrated, shareable power supply system located on at least one side of the helmetless support and ventilation system, wherein the integrated, shareable power supply system is electrically connected to the power module.

2. The integrated, shareable power supply system, according to claim 1, wherein the integrated, shareable power supply system further comprises:
a first power port; and
a second power port located adjacent to the first power port.

3. The integrated, shareable power supply system, according to claim 2, wherein the first power port further comprises:
a lighting power port.

4. The integrated, shareable power supply system, according to claim 2, wherein the second power port further comprises:
an electrical device power port.

5. The integrated, shareable power supply system, according to claim 1, wherein the integrated, shareable power supply system further comprises:
at least one auxiliary battery pack operatively connected to the helmetless support and ventilation system, wherein the at least one auxiliary battery pack is operatively connected to the power module.

6. The integrated, shareable power supply system, according to claim 5, wherein the integrated, shareable power supply system further comprises:
at least one auxiliary battery pack operatively connected to the helmetless support and ventilation system, wherein the at least one auxiliary battery pack is operatively connected to a yoke module.

7. A method of constructing an integrated, shareable power supply system for medical procedure devices, comprising:
providing a helmetless support and ventilation system;
providing a power module that is located within the helmetless support and ventilation system; and
providing an integrated, shareable power supply system that is located on at least one side of the helmetless support and ventilation system, wherein the integrated, shareable power supply system is electrically connected to the power module.

8. The method, according to claim 7, wherein the method further comprises:
providing a first power port; and
providing a second power port that is located adjacent to the first power port.

9. The method, according to claim 8, wherein the first power port further comprises:
a lighting power port.

10. The method, according to claim 8, wherein the second power port further comprises:
an electrical device power port.

11. The method, according to claim 7, wherein the method further comprises:
providing at least one auxiliary battery pack that is operatively connected to the helmetless support and ventilation system, wherein the at least one auxiliary battery pack is operatively connected to the power module.

12. The method, according to claim 11, wherein the method further comprises:
providing at least one auxiliary battery pack operatively connected to the helmetless support and ventilation system, wherein the at least one auxiliary battery pack is operatively connected to a yoke module.

13. A method of using an integrated, shareable power supply system for medical procedure devices, comprising:
providing a helmetless support and ventilation system;
providing a power module that is located within the helmetless support and ventilation system; and
providing an integrated, shareable power supply system that is located on at least one side of the helmetless support and ventilation system, wherein the integrated, shareable power supply system is electrically connected to the power module, and wherein the power module is configured to supply electrical power to the integrated, shareable power supply system.

14. The method, according to claim 13, wherein the method further comprises:
providing a first power port; and
providing a second power port that is located adjacent to the first power port.

15. The method, according to claim 14, wherein the first power port further comprises:
a lighting power port.

16. The method, according to claim 14, wherein the second power port further comprises:
an electrical device power port.

17. The method, according to claim 13, wherein the method further comprises:
providing at least one auxiliary battery pack that is operatively connected to the helmetless support and ventilation system, wherein the at least one auxiliary battery pack is operatively connected to the power module.

18. The method, according to claim 17, wherein the method further comprises:
providing at least one auxiliary battery pack operatively connected to the helmetless support and ventilation system, wherein the at least one auxiliary battery pack is operatively connected to a yoke module.
